(19) United States
(12) Patent Application Publication
(10) Pub. No.: US 2015/0327871 A1
(43) Pub. Date: Nov. 19, 2015

(54) METHODS, SYSTEMS, AND DEVICES FOR TARGETING A RADIAL ACCESS PUNCTURE SITE

(71) Applicant: Abbott Cardiovascular Systems, Inc., Santa Clara, CA (US)

(72) Inventors: Aaron M. Fortson, Fremont, CA (US); Timothy C. Reynolds, Sunnyvale, CA (US)

(73) Assignee: Abbott Cardiovascular Systems, Inc., Santa Clara, CA (US)

(21) Appl. No.: 14/278,972

(22) Filed: May 15, 2014

(51) Int. Cl. A61B 17/132; A61B 17/135

(52) U.S. Cl. CPC A61B 17/1325; A61B 17/135

(57) ABSTRACT

Methods, systems, and devices for applying targeted force to a radial access puncture site to aid in arresting bleeding at the puncture site. Such a device may include a cuff (e.g., band) for placement over a wrist of a patient, and an inflatable balloon attached to the cuff and disposed over the puncture site during use, such that the inflatable balloon applies pressure to the site during use. The device includes targeting indicia disposed on (i) the inflatable balloon, and/or (ii) an optional rigid or semi-rigid member (e.g., as a backing layer for the balloon). The targeting indicia identifies that portion of the balloon to be positioned on the puncture site so that the inflated balloon applies pressure to the site during use. An associated system may include a cuff device, and a fluid source from which an inflation fluid may be delivered into the inflatable balloon during use.

### BACKGROUND

The present invention relates generally to apparatus and methods for the closing of an access passage opened within a body lumen. More particularly, the present invention relates to techniques for closure of arterial and venous puncture sites, particularly, a radial artery access site.

A number of diagnostic and interventional vascular procedures are now performed translumenally. A catheter is introduced into the vascular system at a convenient access location and guided through the vascular system to a target location using established techniques. Such procedures require vascular access, which is usually established during the well-known Seldinger technique. Vascular access is generally provided through an introducer sheath, which is positioned to extend from outside the patient body into the vascular lumen.

Accessing the vasculature through the radial artery, rather than the larger femoral artery has become increasingly popular as the structures employed in such procedures and introduced into the vasculature become smaller. No matter the access site, when vascular access is no longer required, the introducer sheath is removed and bleeding at the puncture site stopped. Various techniques are currently available for providing hemostasis (the cessation of bleeding).

One approach is to use fasteners, suturing, etc. to close the site. Such techniques can be problematic due to the need to target suture capture for retrieval through the artery or other body lumen wall, which often requires complicated mechanisms. In addition, where the site is a radial artery access site, such fastening or closure devices are typically designed for use in larger access sites, such as a femoral access site, and may thus represent “overkill” in closing a radial access site. In addition, such techniques demand a high level of practitioner skill. Finally, even when performed properly, such procedures may still result in minor oozing from the access tract adjacent the site.

Another approach is to apply external force near and upstream from the puncture site, typically by manual or “digital” compression. This approach is time consuming, frequently requiring one-half hour or more of practitioner compression before hemostasis is assured. Additionally, such compression techniques rely on clot formation, which can be delayed until anticoagulants used in vascular therapy procedures (such as for heart attacks, stent deployment, non-optical PTCA results, and the like) wear off. This can take two to four hours, thereby increasing the time required before completion of a typical compression technique. The compression procedure is further often uncomfortable for the patient, frequently requiring analgesics to be tolerable. Moreover, the application of excessive pressure can at times totally occlude the underlying blood vessel, resulting in ischemia thrombosis, and/or nerve damage.

Thus, compression techniques often require significant time of highly trained individuals, while dedicating such personnel to this task is both expensive and inefficient. As such, there continues to be a need for devices and methods that might provide improved closure of radial access sites.

### BRIEF SUMMARY

The present disclosure describes devices, systems and methods for applying targeted force to a radial access puncture site to provide hemostasis and closure of the site. The device may comprise a cuff (e.g., band) for placement over (e.g., around) a wrist of a patient, and an inflatable balloon attached to the cuff and disposed over the radial access puncture site during use. During use, upon inflation, the inflatable balloon applies pressure to the radial access puncture site. The device further includes targeting indicia disposed on at least one of the inflatable balloon or on an optional rigid or semi-rigid member (e.g., the device may optionally include a rigid or semi-rigid member disposed as a backing layer relative to the inflatable balloon). A related system may include such a device, and a fluid source from which an inflating fluid may be delivered into the inflatable balloon associated with the cuff.

The targeting indicia identifies that portion of the balloon to be positioned on the radial access puncture site so that the inflatable balloon applies pressure to the radial access puncture site once inflated. For example, by positioning the indicia over the puncture site, the position and orientation of the inflatable balloon may be precisely positioned for effective hemostasis, as the balloon applies a targeted pressure over the puncture site until hemostasis is achieved. Because the pressure is applied by the balloon and accompanying cuff device of such a device, continuous attendance of a highly trained medical practitioner may not be required.

Another embodiment of a device may include a cuff for placement over a wrist of a patient, an inflatable balloon attached to the cuff and disposed over the radial access puncture site during use, such that the balloon applies pressure to the puncture site during use, a rigid or semi-rigid member disposed between the cuff and the inflatable balloon, and targeting indicia disposed on both the inflatable balloon and the rigid or semi-rigid member. The targeting indicia identifies that portion of the balloon to be positioned over the puncture site so that the balloon applies pressure to the puncture site once inflated, during use.

Another embodiment of the present disclosure is directed to a method for applying targeted force to a radial access puncture site. The method may include placing a cuff of a device for applying targeted force over a wrist of a patient. The device may include a cuff for placement over a wrist of a patient, and an inflatable balloon structure attached to the cuff that is disposed over the radial access puncture site during use. The device further includes targeting indicia disposed on at least one of the inflatable balloon or on an optional rigid or semi-rigid member disposed as a backing layer relative to the inflatable balloon. The targeting indicia identifies that portion of the balloon that should be positioned over the puncture site so that the inflatable balloon applies pressure to the puncture site once inflated. The structures of the cuff device (e.g., the cuff, the balloon structure, a rigid or semi-rigid member, etc.) may be adjusted in position to position the targeting indicia over the puncture site. The inflatable balloon structure may be inflated with an inflating fluid, so that the inflated balloon applies pressure to the puncture site to provide hemostasis of the puncture site while limiting risk of damage to nerves adjacent the puncture site.

These and other objects and features of the present disclosure will become more fully apparent from the following description and appended claims, or may be learned by the practice of the embodiments of the invention as set forth hereinafter.

### BRIEF DESCRIPTION OF THE DRAWINGS

To further clarify the above and other advantages and features of the present disclosure, a more particular description of the invention will be rendered by reference to specific embodiments thereof which are illustrated in the appended drawings. It is appreciated that these drawings depict only illustrated embodiments of the invention and are therefore not to be considered limiting of its scope. Embodiments of the invention will be described and explained with additional specificity and detail through the use of the accompanying drawings in which:

FIG. 1A is a schematic perspective view of an exemplary system including a cuff device with targeting indicia for placement over a radial access puncture site;

FIG. 1B is a schematic perspective view of the system of FIG. 1A, with the cuff device placed over the puncture site of the patient;

FIGS. 1C and 1D are schematic perspective views of another cuff device, with FIG. 1C showing the cuff device before placement over the puncture site, and FIG. 1D showing the cuff device placed over the puncture site;

FIG. 2A is a cross-sectional view of a portion of the exemplary cuff device of FIGS. 1A-1B, with the balloon at least partially deflated;

FIG. 2B is a cross-sectional view similar to that of FIG. 2A, with the balloon of the cuff device inflated;

FIG. 2C is a cross-sectional view similar to that of FIG. 2B, with the balloon of the cuff device inflated and positioned against the wrist, the targeting indicia on the balloon and rigid or semi-rigid member aligned with one another and the puncture site;

FIG. 2D is a cross-sectional view similar to that of FIG. 2C, but illustrating another configuration for attachment of the balloon to the optional rigid or semi-rigid member; and

FIG. 2E is a cross-sectional view similar to that of FIG. 2C, illustrating another configuration.

### DETAILED DESCRIPTION

### I. Introduction

In one aspect, the present disclosure describes methods, systems, and devices that may be included as part of such systems for applying targeted force to a radial access puncture site to arrest bleeding at the puncture site. Such a device may include a cuff (e.g., band) for placement over a wrist of a patient, and an inflatable balloon attached to the cuff and disposed over the radial access puncture site during use, such that the inflatable balloon applies pressure to the radial access puncture site during use. The device includes targeting indicia disposed on at least one of (i) the inflatable balloon, or (ii) a rigid or semi-rigid member that may optionally be provided (e.g., as a backing layer for the balloon). The targeting indicia identifies that portion of the balloon to be positioned on the puncture site so that the inflatable balloon applies pressure to the puncture site once inflated during use. An associated system may include a cuff device, and a fluid source from which an inflation fluid may be delivered into the inflatable balloon during use.

### II. Exemplary Devices and Methods

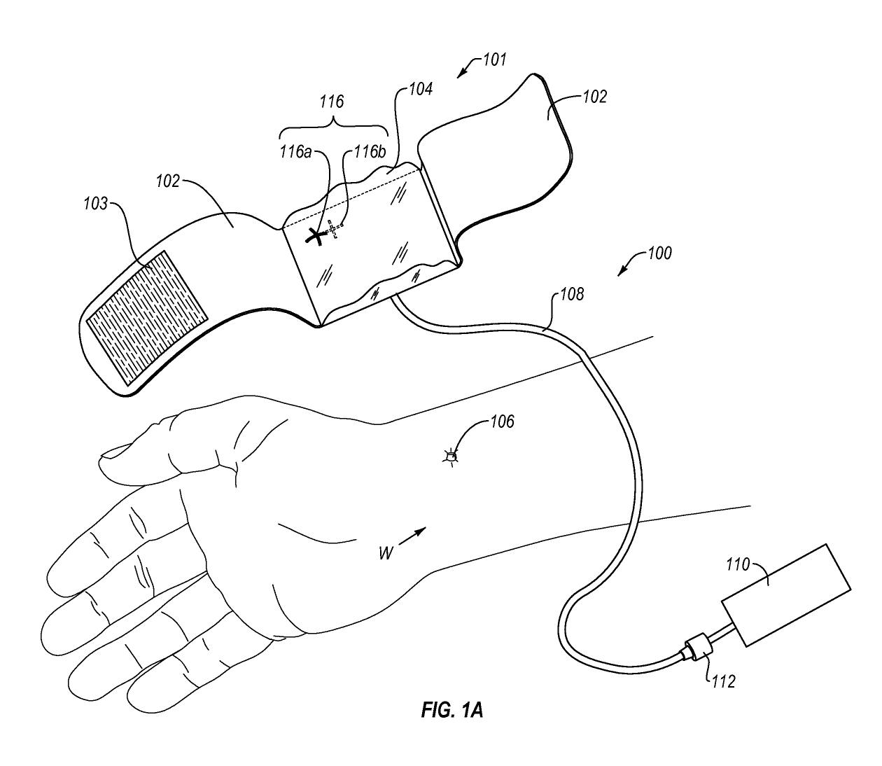

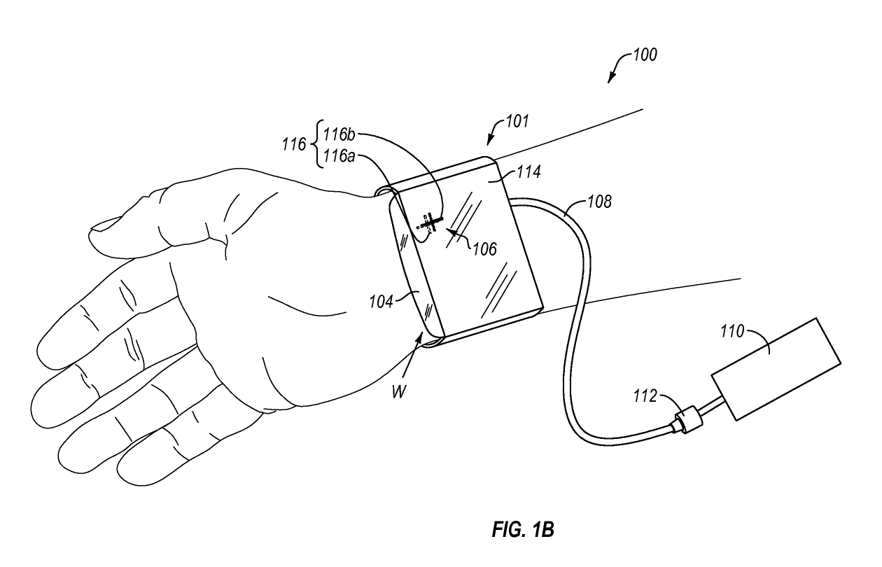

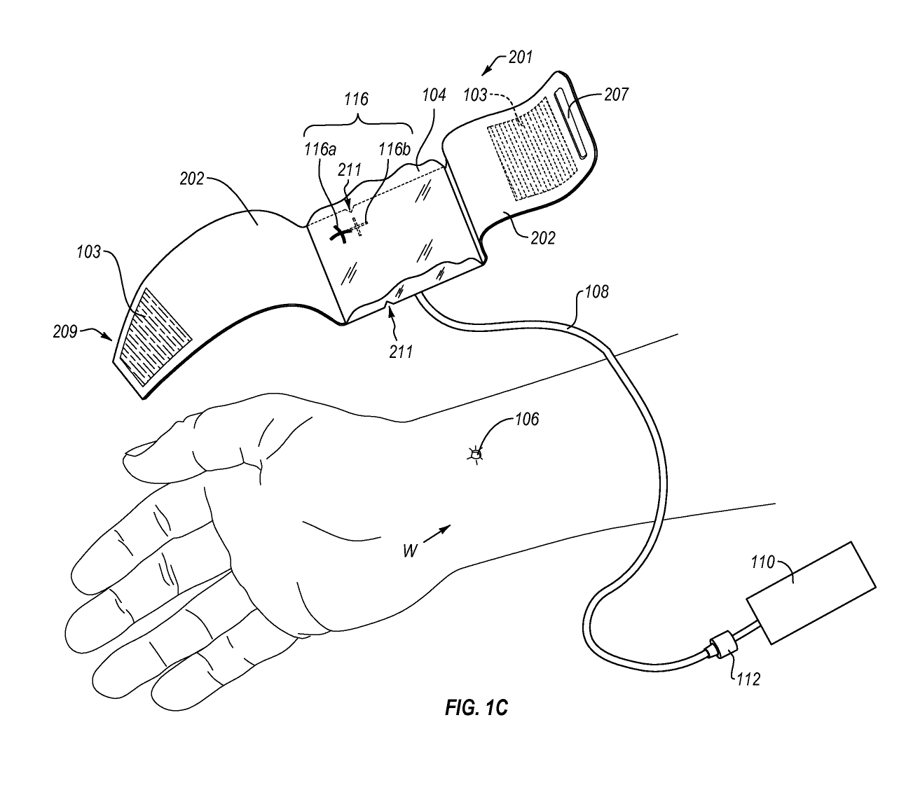

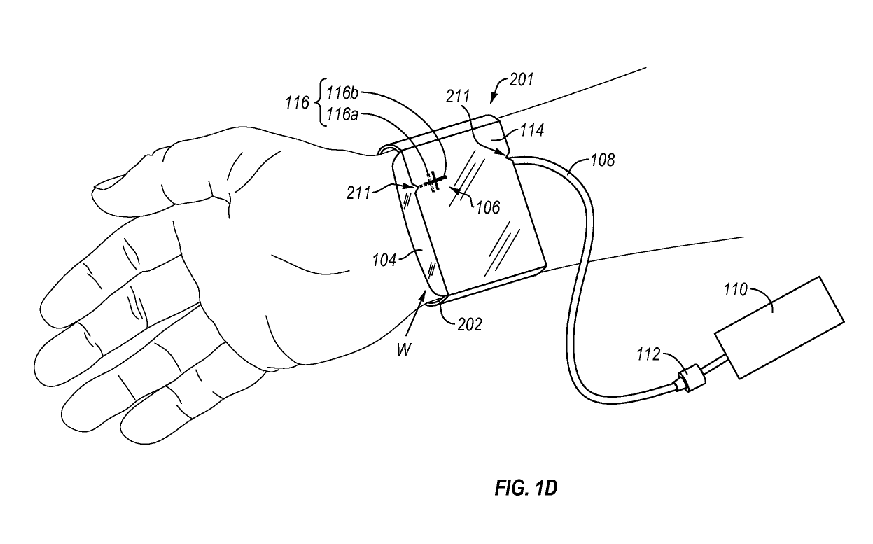

Referring to FIGS. 1A-1B, an exemplary system 100 including device 101 is shown, including a cuff 102 that can be placed over the wrist W of a patient. Cuff 102 may be configured as any suitable structure that can be disposed over (e.g., around) a wrist of a patient. For example, cuff 102 may comprise an elongate band, as seen in FIG. 1A that may be wrapped over and secured to itself (e.g., similar to a blood pressure cuff). For example, cuff 102 may include any suitable fastener for securing the cuff 102 around the patient's wrist, such as snaps, hook and loop fasteners (e.g., VELCRO), a buckle, etc. FIG. 1A illustrates one part of hook and loop fastener (e.g., hooks 103) on one face of band 102, while the other portion of the hook and loop mechanism (e.g., loops) may be disposed on a corresponding face of another portion of band 102. Of course, other fastening mechanisms may alternatively be employed.

In another embodiment, the cuff 102 may be configured as a continuous round band (e.g., generally circular or other hollow rounded shape) that is received over the wrist. For example, the cuff 102 could comprise an elastic material that would allow it to be expanded as it is advanced over the hand, where it retracts to its original un-expanded configuration once over the wrist. Such a continuous round band could be provided by attaching opposed ends of an elongate band (e.g., as seen in FIG. 1A) to one another using a suitable fastening mechanism (e.g., stitched together, VELCRO, etc.). Such a band may be elastic. FIG. 1A shows radial access puncture site 106, just as cuff 102 is being prepared for placement thereover. The inflatable balloon 104 may be disposed on cuff 102 so as to be positioned over puncture site 106 as cuff 102 is tightened around wrist W. Device 101 further includes targeting indicia 116 disposed on one or both of inflatable balloon 104, or rigid or semi-rigid member 114. FIG. 1A shows targeting indicia 116a and 116b on both balloon 104 and rigid or semi-rigid backing member 114, respectively. It will be appreciated that in an embodiment, only one of indicia 116a or 116b may be provided. Providing indicia on both balloon 104 and member 114 may provide a further advantage as the indicia 116a and 116b become aligned (e.g., axially) with puncture site 106, as balloon 104 is inflated, as explained in further detail below. FIG. 1B illustrates band 102 once it has been wrapped around the wrist W, with balloon 104 (e.g., and indicia 116) positioned over puncture site 106, so as to apply pressure thereto.

Inflatable balloon structure 104 may be attached to cuff 102, where balloon 104, and particularly indicia 116 is positioned over the radial access puncture site 106 (e.g., somewhat above the center of the wrist W on the inside of the arm), as seen in FIGS. 1A-1B. FIG. 1B shows balloon 104 positioned over radial artery puncture site 106 of the right arm. It will be apparent that a similar cuff and balloon could be positioned over a radial artery puncture site on the left arm. Inflation of the balloon structure 104 causes application of pressure to puncture site 106, so as to compress site 106 and lead to hemostasis, closing puncture site 106. In addition to cuff 102 and balloon 104, system 100 may further include a fluid delivery line 108, a fluid source 110 (shown schematically), and a valve 112 which may be in fluid communication (e.g., selective fluid communication) with balloon 104. An inflation fluid (e.g., air, saline, water, etc.) may be delivered from source 110, through valve 112, through line 108, and into balloon 104 of device 101, inflating balloon 104, which inflation fluid may exert the desired pressure to puncture site 106. Fluid source 110 may comprise a syringe or other suitable structure for containing and/or compressing the fluid. Correct positioning of balloon 104 over site 106 may be aided by the presence of targeting indicia 116 on balloon 104 (e.g., indicia 116a) and/or rigid or semi-rigid member 114 (e.g., indicia 116b).

Balloon 104 may be any suitable inflation structure, e.g., that can be inflated upon introduction of an inflation fluid. Inflation of balloon 104 may be achieved using fluid source 110 (e.g., a syringe or other suitable source). In an embodiment, inflation fluid may be delivered through line 108 to balloon 104. In another embodiment, a syringe or other fluid source may be coupled to balloon 104 directly, without an intervening fluid delivery line 108. In an embodiment, a liquid inflation fluid (e.g., water, saline, etc.) is employed, which may provide greater pressure to puncture site 106 than a gaseous fluid (e.g., air, nitrogen, etc.).

In an embodiment, the inflation fluid may be cooled below ambient temperature, to provide cooling to puncture site 106, e.g., to limit inflammation and/or accelerate hemostasis at the site. In another embodiment, cooling may be provided by passing a cooling fluid separate from the inflation fluid through channels formed within the inflation balloon, cuff, rigid or semi-rigid member that backs the balloon, etc. Such cooling channels may be configured to provide separation between the inflation fluid and any cooling fluid. An ice pack or other cooling pack could also be included within the cuff device (e.g., a compartment in the cuff device that receives and retains an ice or cooling pack). In other embodiments, heating could alternatively be provided through similar mechanisms (e.g., heating the inflation fluid, a separate heating fluid, a heating pack, etc.), where heating may be desirable.

In addition, where desired, a hemostatic or coagulating agent may be applied to the puncture site and/or the balloon 104 (e.g., to that portion of balloon 104 that may contact puncture site 106, on indicia 116a, etc.). Examples of such hemostatic and coagulating agents include, but are not limited to aluminum compounds, iron-based compounds, and combinations thereof. Specific examples of such include, but are not limited to potassium aluminum sulfate, aluminum ammonium sulfate, aluminum sulfate, aluminum chlorohydrate, aluminum acetate, aluminum chloride, ferric sulfate, ferric subsulfate, ferric chloride, and mixtures thereof. Other such agents may include permanganates, tannins, zinc chloride, chitosan, etc. In one embodiment a vasoconstrictor such as epinephrine and/or propylhexedrine may also be provided. Such agents may be coated on the balloon, or delivered through pores in an outer layer of the balloon, etc. for delivery to puncture site 106.

In an embodiment, system 100 or device 101 may be provided in a single sterilized kit (e.g., a package including the cuff, attached balloon, fluid delivery lines, fluid source, etc.) In another embodiment, the cuff and balloon device 101 may be employed with existing tubing, syringes, other fluid sources, etc. commonly found in a catheterization lab, hospital, or similar clinical facility.

FIGS. 1C-1D illustrate another cuff device embodiment 201 that may be similar to device 101 and cuff 102 shown in FIGS. 1A-1B, but is shown as including a slot 207 disposed in an end of strap or cuff 202, allowing an opposite end 209 to be inserted through slot 207, and cinched and secured (e.g., with VELCRO 103, or any other suitable mechanism) as seen in FIG. 1D. VELCRO 103 is shown on opposed surfaces of the strap, so as to be oriented to face one another once cuff 202 is disposed around wrist W. As shown, end 209 which is inserted through slot 207 may be tapered, so as to facilitate easier insertion into slot 207. Slot 207 may be formed in the cuff 202, and/or through a rigid or semi-rigid member on which balloon 104 is disposed. It will be appreciated that a wide variety of cuff configurations will be suitable for use with the present invention. As such, it will be appreciated that any cuff device may include the features described below or elsewhere herein.

Cuff device 201 is further shown as including alignment notches 236 on one or both peripheral edges of the elongate cuff 202, formed in the perimeter edge of a rigid or semi-rigid member 114 which acts as a backing member for balloon 104. Notches 236 may be positioned so that the vertex of notches 211 are aligned with one another, and with the portion of inflatable balloon 104 (e.g., indicia 116b) to be positioned over puncture site 106. Such notches 211 may further aid the practitioner in correctly placing and positioning cuff 202. It will be appreciated that markings visible on the perimeter edge, rather than necessarily notches, may be employed for the same purpose. Of course, both may be provided (e.g., notches with marked or colored edges). One or both of respective ends of strap shaped cuff 202 may include self-adhesive, VELCRO 103, or other suitable mechanism for attachment of the opposed ends together.

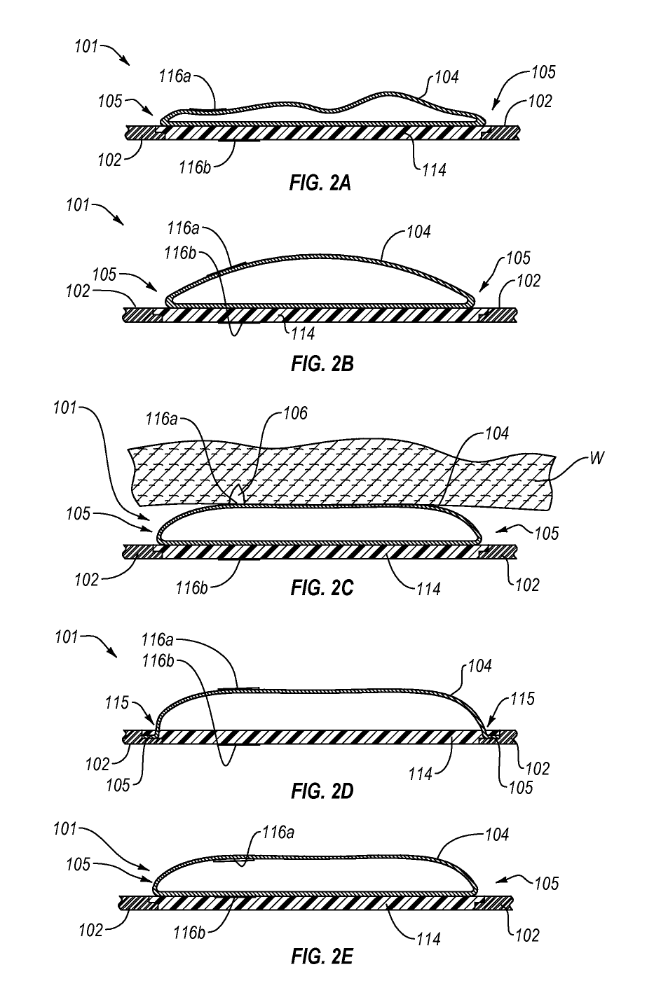

FIGS. 2A-2C illustrate close up cross-sectional views of a portion of an exemplary cuff device 101, which may be included in such a system 100. For example, as seen in FIGS. 2A-2C, system 100 and device 101 may include a rigid or semi-rigid (e.g., more rigid than balloon 104 and/or cuff 102) member 114 on which balloon 104 may be disposed. Such a rigid or semi-rigid member may comprise a sheet of plastic, metal, paperboard, glass or other material on which balloon 104 may be mounted, providing a backing layer providing support for balloon 104. In an embodiment, at least a portion of rigid or semi-rigid member 114 may be formed of a transparent or translucent material (e.g., plastic, glass, etc.), as one or more of indicia 116 may be disposed on member 114, and the ability to see through member 114 as indicia 116 is aligned over puncture site 106 may be particularly advantageous. At least a portion of balloon 104 may similarly be transparent or translucent. For example, at least those portions of member 114 and balloon 104 surrounding indicia 116 may be transparent or translucent. Member 114 provides an increased degree of rigidity as compared to the balloon 104 (e.g., comprising a thin polymeric film) and/or cuff 102 (e.g., comprising a flexible fabric or similar material). Such a rigid or semi-rigid member 114 may serve to limit movement of balloon 104 in a direction opposite a direction of force applied to puncture site 106 (e.g., in a direction away from puncture site 106). For example, where provided, rigid member 114 may provide a structure which balloon 104 may “push against” to apply the desired force to puncture site 106 (e.g., see FIG. 2C).

Member 114 may be localized (e.g., smaller than cuff 102 and/or balloon 104), or may generally match the configuration or surface area (e.g., footprint) covered by balloon 104. For example, rigid or semi-rigid member 114 may extend to or past an outer peripheral edge 105 of balloon 104. In another embodiment, the outer peripheral edge of member 114 may be disposed within the perimeter defined by balloon 104 (e.g., being localized to the puncture site 106 and immediate surrounding tissue). For example, in such an embodiment, the footprint of balloon 104 may be larger than that of member 114. In the illustrated embodiment, rigid or semi-rigid member 114 is configured so as to be a backing support layer on which balloon 104 is disposed.

Cuff 102 may include multiple discontinuous portions, with member 114 disposed therebetween, as seen in FIGS. 2A-2C. Such an embodiment allows viewing through a transparent member 114 and balloon 104, so as to allow a practitioner to visualize alignment between indicia 116 (e.g., indicia 116a, 116b), and puncture site 106.

FIG. 2D illustrates another configuration, where peripheral edge 105 of balloon 104 may be received through a slot 115 that may be disposed near a peripheral edge 105 of member 114. Such a configuration may employ member 114 to aid in securing edge 105 of balloon 104 in place (e.g., sandwiching edge 105 between a portion of member 114 and cuff 102). In FIGS. 2A-2C, balloon 104 is shown as defining a closed shape itself, defining the inflatable balloon volume. In FIG. 2D, balloon 104 is shown as relying on rigid or semi-rigid backing member 114 to close the inflatable balloon volume. All such illustrations are exemplary, and it will be appreciated that features of one embodiment may be incorporated into another embodiment. For example, the embodiment of FIGS. 2A-2C could employ member 114 in providing the closed inflatable shape of balloon 104, or the embodiment of FIG. 2D could include a balloon wall thickness across member 114, as FIGS. 2A-2C.

In the embodiment illustrated in FIGS. 2A-2C, balloon 104 is shown attached indirectly to cuff 102, as balloon 104 is attached to rigid or semi-rigid member 114, which member 114 is in turn attached to cuff 102. Any suitable mechanism may be employed for such attachment. For example, one structure may be attached to another structure using stitching, adhesives, buttons, snaps, or any other suitable fastener. In an embodiment, rigid or semi-rigid member 114 may be stitched into cuff 102, and balloon 104 may be attached to member 114 using a suitable adhesive.

While shown with a rigid or semi-rigid member 114, it will be appreciated that in another embodiment no such member 114 may be present. For example, balloon 104 may be attached directly to cuff 102, without any member 114, and indicia 116 may be provided on balloon 104. In an embodiment, such a cuff may be transparent or translucent to allow viewing therethrough. In another embodiment, the cuff could be discontinuous or include a hole or window therethrough, allowing viewing of the indicia on balloon 104. Even where a rigid or semi-rigid member 114 is present, in an embodiment, at least a portion of balloon 104 may be directly attached to cuff 102 (e.g., as seen in FIG. 2D, or where member 114 is smaller (i.e., smaller in footprint) than balloon 104, being localized to a very small area over puncture site 106, while balloon 104 may cover a larger area. In such an embodiment, viewing of the area surrounding indicia 116 may be possible where at least the surrounding portions of member 114 and balloon 104 are transparent or translucent, even where cuff 102 may not be transparent or translucent.

Balloon 104 may be particularly configured to apply a compression force to puncture site 106, while indicia 116 (e.g., cross-hairs as shown, or other markings, such as a bulls-eye, target, a letter, symbol, or other character) may aid in positioning balloon 104 and cuff 102 so as to target the applied force to site 106. For example, in accessing the radial artery through a radial access puncture site, a relatively small needle (e.g., about 0.03 inch in outside diameter) may be used, and introducer wires, catheters, sheaths, and other structures employed in forming the small puncture site and initiating access to the radial artery may be typically quite small (e.g., up to about 0.04 or 0.05 inch in outside diameter). A catheter sheath introduced into the radial artery may be somewhat larger, but still significantly smaller than those introduced into the femoral artery, as the radial artery is much smaller. For example, such a sheath may be up to about 6 Fr (about 2 mm, or about 0.08 inch) in outside diameter. Because the puncture site is typically relatively small in area, bleeding from puncture site 106 may be arrested through compression, and the patient may even be allowed to stand and walk soon after the intravascular procedure. This is in sharp contrast to closure after femoral access, where the patient may be required to remain still for several hours (e.g., at least about 5 hours) after the intravascular procedure is complete.

Balloon 104 may be configured to apply pressure to puncture site 106 and a small adjacent area surrounding puncture site 106 on wrist W. For example, in an embodiment, system 100 and device 101 may apply targeted pressure to a region having an area of about 5 in² or less, 3 in² or less, about 2 in² or less, about 1 in² or less, about 0.75 in² or less, or about 0.5 in² or less. The magnitude of applied pressure may be sufficient to provide hemostasis and closure of the puncture site, while not being so great as to cause nerve damage.

By providing indicia 116 on both balloon 104 and rigid or semi-rigid backing member 114, the practitioner may be provided an indication as to when the appropriate magnitude of pressure is achieved by alignment of indicia 116a, 116b and puncture site 106. For example, As seen in FIG. 2A, when balloon 104 is deflated or only partially inflated, indicia 116a and 116b may not be aligned (e.g., coaxially) with one another, or with puncture site 106. As inflation progresses (e.g., FIG. 2B), indicia 116a on balloon 104 may move towards alignment over indicia 116b on member 114, which may already be aligned with puncture site 106.

Indicia 116a may be particularly provided so that upon inflation of balloon 104 to a level where indicia 116a becomes aligned with puncture site 106 (e.g., and indicia 116b where indicia is also provided on member 114), an appropriate magnitude of force is applied to site 106 so as to result in the desired hemostasis and closure of puncture site 106. By providing a configuration where alignment (e.g., coaxial alignment between site 106 and one or more indicia 116) corresponds to application of an appropriate force level, risk of nerve damage due to application of excessive compressive force may be reduced. FIG. 2C illustrates such coaxial alignment between indicia 116a, 116b, and site 106. In an embodiment, at least a portion of the compressive force may be applied to the radial artery at a location “upstream” from puncture site 106. For example, even where the user aligns indicia 116 with site 106 (which presents a convenient “landmark”), application of the actual compressive force may at least in part be directed to a location that is upstream from site 106.

As seen in FIG. 2C, with balloon 104 pressed against wrist W and indicia 116a and/or 116b aligned over site 106, the user may know that the appropriate level of inflation has been reached, and corresponding appropriate magnitude of force is being applied. As is apparent from FIGS. 2C and 1B, being able to see through the portion of member 114 surrounding indicia 116b and the portion of balloon 104 surrounding indicia 116a may be greatly advantageous in positioning, inflating, and securing device 101 about wrist W. Cuff 102 may be wrapped or otherwise placed over the wrist of the patient, and secured to itself (e.g., by VELCRO 103 or similar fastener mechanisms). Because of the targeted compressive force applied by balloon 104 to site 106, hemostasis may be quickly achieved, closing puncture site 106.

Inflation of balloon 104 may be achieved by introducing a suitable inflation fluid from a fluid source 110 (e.g., a syringe or other source) directly into balloon 104, or through delivery line 108, inflating balloon 104 so as to cause balloon 104 to extend from cuff 102 and compress against wrist W, particularly site 106.

Indicia 116 may include any marking (e.g., symbol, character, etc.) or other indication (e.g., providing a portion of balloon 104 with a color that differs as compared to adjacent portions of balloon 104) providing a visually differentiable “landmark” to be aligned over puncture site 106 that can be referenced by a practitioner during placement and use. For example, the balloon 104 may include regions of different colors (e.g., colored (e.g., red, green, blue, yellow, pink, white, etc. in one portion, and another color or clear in another portion) to aid in targeting. Colored portions may be lightly tinted, so as to still be transparent or translucent, allowing a practitioner to see therethrough. The vicinity surrounding one or more of indicia 116 may be of a color that differs from the rest of the balloon. In an embodiment, indicia 116a and 116b may be of different colors, so that when they align, a third color results. For example, where one is blue, and one is yellow, and on alignment, the aligned indicia appears green, or one is blue, and one is red, and on alignment, the aligned indicia appears purple.

In an embodiment, the balloon may exhibit one color under a first inflation pressure and another color under a second inflation pressure, the second inflation pressure signifying a desired pressure to aid with hemostasis while limiting risk of damage to nerves adjacent the puncture site. For example, a lighter shade of color, or greater degree of transparency or translucency (e.g., red to pink, or opaque red to transparent/translucent red) may be apparent at the second, higher inflation pressure as compared to a first (e.g., nearly deflated) pressure. Upon visually seeing the second color, lighter color shade, or greater degree of transparency/translucency (all referred to herein as a second color for simplicity), the practitioner knows that the desired pressure is being applied.

While two indicia 116 are shown, one on balloon 104 and one on member 114, it will be appreciated that a similar coaxial alignment may be achieved with placement of indicia 116a on an upper surface of balloon 104 as shown, and placement of indicia 116 on a lower surface of the balloon 104. FIG. 2E shows such an embodiment.

The geometry of balloon 104 may be as desired. For example, in an embodiment, balloon 104 may cover about 50% or more of the inside of wrist W, as seen in FIG. 1B. FIG. 1B illustrates an example where the entire or nearly the entire surface area of wrist W is covered by balloon 104, which may have a width approximately equal to the adjacent portions of cuff 102, as shown. In another embodiment, balloon may have a width that is greater or less than adjacent portions of cuff 102.

In an embodiment, balloon 104 may have a size and shape so as to be disposed only over a small area surrounding puncture site 106, rather than the entire wrist W. For example, typically, the radial access puncture site is disposed in an upper left quadrant of the wrist W (or upper right for a wrist of a left hand). As such, balloon 104 may simply cover this portion of the wrist, surrounding the puncture site, rather than the entire, or a large fraction (e.g., 50% or more) of wrist W. In any case, balloon 104 may be such so as to correspond to the position of the radial access puncture site, as cuff 102 is placed over wrist W. The balloon 104 may represent only a relatively small fraction of the overall surface area of wrist W as a whole, so as to apply force to only a small target area at the puncture site 106. For example, balloon 104 may have a footprint that is less than about 50%, less than about 40%, less than about 30%, less than about 25%, or less than about 20% of the surface area of the inside surface of wrist W.

The geometric shape of balloon 104 may be of any desired shape. For example, while FIGS. 1A-1B show a generally rectangular or square shape, it will be appreciated that various other shapes may be employed, e.g., circular, square, oval, other rounded shape, other closed curve shape, regular or irregular polygonal shaped, other closed geometric shape, etc.

The balloon 104 as a whole may have a size that is not larger than so as to cover the inside surface of the wrist (e.g., without wrapping around towards the front of the arm). For example, it may be not more than about 1 inch wide and about 2 inches long. As described above, alternatively, the balloon may cover only a small fraction of the inside surface of the wrist W, which is generally planar. The rigid or semi-rigid member 114 may be similarly sized and shaped as a generally planar sheet-like member, particularly where its rigidity may prevent it from readily wrapping around the arm towards the front of the arm. In another embodiment, member 114 may include a non-planar portion, pre-shaped with a curvature to accommodate curvature of the wrist associated with the transition from the inside of the wrist towards the opposite face of the wrist.

Placement of indicia 116 on balloon 104 and/or member 114 may be as desired. For example, particularly where at least those portions of balloon and member 114 surrounding indicia 116a and 116b, respectively, are transparent or translucent, indicia may be disposed on an outside and/or inside surface of balloon 104 and/or member 114, as desired. For example, FIGS. 2A-2C, showing progressive inflation and positioning of balloon 104 show indicia 116a on an outside surface of balloon 104, and indicia 116b on an exterior surface of member 114. It will be appreciated that alternative placement is also possible. In an embodiment, one or more of indicia 116 may be embedded or encapsulated (e.g., sandwiched) between laminating layers of balloon 104 or member 114. For example, such placement (or placement on the inside, un-exposed surface of balloon 104 or member 114) may serve to protect indicia 116 from being worn away or otherwise damaged during use. Such an embodiment may be particularly beneficial where device 101 may be sterilized and reused. FIG. 2E shows an embodiment with indicia 116a on an inside surface of balloon 104, and indicia 116b on the lower surface of balloon 104, sandwiched between balloon 104 and member 114.

The system and device may be specifically configured to apply targeted pressure over the relatively small puncture site, and optionally a small area surrounding the puncture site, so as to achieve hemostasis and close puncture site 106, while limiting unneeded pressure so as to reduce risk of nerve damage or other unwanted complications or side-effects. Embodiments including features as part of the balloon or other portions of such a device or system, such features applying a targeted force, are disclosed in a U.S. patent application Ser. No. ______ bearing attorney Docket No. 16497.260, filed the same day as the present application, and herein incorporated by reference in its entirety. Such features may include, but are not limited to (i) providing the balloon with varying wall thickness, with a thinner-walled portion that applies a focused pressure to the puncture site, (ii) two balloons where one applies focused force, or (iii) an outer sheath with a hole placed over the balloon so that a portion of the balloon protrudes through the hole and applies focused force. In such embodiments, the targeting indicia may be disposed on or over the feature providing focused, targeted force (e.g., (i) on the thin-walled portion of the balloon, (ii) on the (e.g., smaller) balloon applying focused force, or (iii) on the portion of the balloon that protrudes through the hole of an accompanying sheath. The targeting indicia on the balloon or other portions of the cuff device described herein could be thickened, shaped or stiffened (e.g., relative to structure adjacent to the indicia) to apply focused pressure, e.g., similar to the embodiments described in application Ser. No. ______ Docket No. 16497.260.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A device for targeting force applied to a radial access puncture site, the device comprising:
a cuff for placement over a wrist of a patient;
an inflatable balloon attached to the cuff and disposed over the radial access puncture site during use, such that the inflatable balloon applies pressure to the radial access puncture site during use;
targeting indicia disposed on at least one of (i) the inflatable balloon or (ii) a rigid or semi-rigid member, the targeting indicia identifying that portion of the balloon to be positioned on the radial access puncture site so that the inflatable balloon applies pressure to the radial access puncture site once inflated during use.

2. The device of claim 1, wherein the device comprises a rigid or semi-rigid member on which the inflatable balloon is disposed.

3. The device of claim 2, wherein targeting indicia are disposed on both a surface of the balloon and a surface of the rigid or semi-rigid member, the balloon and rigid or semi-rigid member being transparent to allow a practitioner to visualize the targeting indicia and the radial access puncture site through the balloon and rigid or semi-rigid member.

4. The device of claim 3, wherein the targeting indicia are not aligned with one another before inflation of the balloon, the targeting indicia becoming axially aligned with one another and the radial access puncture site once the balloon is inflated adjacent a wrist of a patient.

5. The device of claim 3, wherein the two targeting indicia are of different colors so that upon alignment of the targeting indicia on the balloon with the targeting indicia on the rigid or semi-rigid member, the aligned targeting indicia appear to be of a third color.

6. The device of claim 2, wherein targeting indicia are disposed on an upper surface of the balloon and on a lower surface of the balloon, the balloon and rigid or semi-rigid member being transparent to allow a practitioner to visualize the targeting indicia and the radial access puncture site through the balloon and rigid or semi-rigid member.

7. The device of claim 1, wherein the balloon includes regions of different colors to aid with targeting the radial access site.

8. The device of claim 1, wherein the balloon has one color under a first inflation pressure and another color under a second inflation pressure, the second inflation pressure signifying a desired pressure to aid with hemostasis while limiting risk of damage to nerves adjacent the puncture site.

9. A device of claim 1, wherein the balloon includes regions of different colors to aid with targeting the radial access site.

10. A system comprising the device of claim 1, further comprising a fluid source from which an inflating fluid may be delivered into the inflatable balloon associated with the cuff during use.

11. A device for targeting force applied to a radial access puncture site, the device comprising:
a cuff for placement over a wrist of a patient;
an inflatable balloon attached to the cuff and disposed over the radial access puncture site during use, such that the inflatable balloon applies pressure to the radial access puncture site upon inflation by a fluid source during use;
a rigid or semi-rigid member disposed between the cuff and the inflatable balloon; and
targeting indicia disposed on both (i) the inflatable balloon and (ii) the rigid or semi-rigid member, the targeting indicia identifying that portion of the balloon to be positioned over the radial access puncture site so that the inflatable balloon applies pressure to the radial access puncture site once inflated during use.

12. The device of claim 11, wherein the balloon and rigid or semi-rigid member are transparent to allow a practitioner to visualize the targeting indicia and the radial access puncture site through the balloon and rigid or semi-rigid member.

13. The device of claim 11, wherein the targeting indicia are not aligned with one another before inflation of the balloon, the targeting indicia becoming axially aligned with one another and the radial access puncture site once the balloon is inflated.

14. The device of claim 11, wherein the two targeting indicia are of different colors so that upon alignment of the targeting indicia on the balloon with the targeting indicia on the rigid or semi-rigid member, the aligned targeting indicia appear to be of a third color.

15. The device of claim 11, wherein the balloon has one color under a first inflation pressure and another color under a second inflation pressure, the second inflation pressure signifying a desired pressure to aid with hemostasis while limiting risk of damage to nerves adjacent the puncture site.

16. The device of claim 11, wherein the targeting indicia are not disposed on an exposed exterior surface of the balloon or rigid or semi-rigid member to protect the indicia from being worn away or otherwise damaged.

17. The device of claim 16, wherein the targeting indicia are disposed on an inner surface of the balloon or rigid or semi-rigid member.

18. A system comprising the device of claim 11, further comprising a fluid source from which an inflating fluid may be delivered into the inflatable balloon associated with the cuff during use.

19. A method for applying targeted force to a radial access puncture site, the method comprising:
placing a cuff of a device for applying targeted force over a wrist of a patient, the device including:
a cuff for placement over a wrist of a patient;
an inflatable balloon attached to the cuff that is disposed over the radial access puncture site during use, such that the inflatable balloon applies pressure to the radial access puncture site during use; and
targeting indicia disposed on at least one of (i) the inflatable balloon or (ii) an optional rigid or semi-rigid member disposed between the cuff and the inflatable balloon, the targeting indicia identifying that portion of the balloon to be positioned over the radial access puncture site so that the inflatable balloon applies pressure to the radial access puncture site once inflated during use.
adjusting the position of one or more of the cuff, inflatable balloon, or the optional rigid or semi-rigid member to position the targeting indicia over the radial access puncture site;
inflating the balloon with an inflating fluid, the inflated balloon applying pressure to the radial access puncture site to provide hemostasis of the puncture site while limiting risk of damage to nerves adjacent the puncture site.

20. The method of claim 19, wherein the device includes the rigid or semi-rigid member, wherein at least a portion of the rigid or semi-rigid member and at least a portion of the balloon are transparent to allow a practitioner to visualize the targeting indicia and the radial access puncture site through the balloon and rigid or semi-rigid member.

21. The method of claim 20, wherein the targeting indicia are not aligned with one another before inflation of the balloon, the targeting indicia becoming axially aligned with one another and the radial access puncture site once the balloon is inflated.

22. The method of claim 19, wherein the inflating fluid is cooled below ambient temperature to cool the puncture site as the inflated balloon applies pressure to the puncture site.
